(19) United States
(12) Patent Application Publication
(10) Pub. No.: US 2006/0240418 A1
(43) Pub. Date: Oct. 26, 2006

(54) Canine gene microarrays

(76) Inventors: James C Diggans, Gaithersburg, MD (US); Mark Porter, Gaithersburg, MD (US); Tao Wei, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/513,369

(22) PCT Filed: May 5, 2003
(86) PCT No.: PCT/US03/13853

§ 371 Date: May 10, 2005

Related U.S. Application Data
(60) Provisional application No. 60/377,240, filed on May 3, 2002.

(51) Int. Cl. C12Q 1/68; G06F 19/00; C07H 21/04

(52) U.S. Cl. 435/6; 536/24.3; 702/20

(57) ABSTRACT

The present invention is based on the identification of novel canine nucleic acid sequences and the construction of canine microarrays containing a significant portion of the canine genome. The microarrays specifically hybridize to canine nucleic acid samples and may be used in drug screening and toxicity assays.

### RELATED APPLICATIONS

This application claims priority under 35 U.S.C. §119(e) to U.S. Provisional Application 60/377,240, filed May 3, 2002, which is herein incorporated by reference in its entirety.

### SEQUENCE LISTING SUBMISSION ON COMPACT DISC

The Sequence Listing submitted concurrently herewith on compact disc under 37 C.F.R. §§1.821(c) and 1.821(e) is herein incorporated by reference in its entirety. Four copies of the Sequence Listing, one on each of four compact discs are provided. Copy 1, Copy 2 and Copy 3 are identical. Copies 1, 2 and 3 are also identical to the CRF. Each electronic copy of the Sequence Listing was created on May 2, 2002 with a file size of 8868 KB. The filenames are as follows: Copy 1-g15116wo.txt; Copy2-g15116wo.txt; Copy 3-g15116wo.txt; CRF-g15116wo.txt.

### BACKGROUND OF THE INVENTION

The need for methods of assessing the impact, including toxicity, of a compound, pharmaceutical agent or environmental pollutant on a cell or living organism has led to the development of procedures which utilize living organisms as biological monitors. The simplest and most convenient of these systems utilize unicellular microorganisms such as yeast and bacteria, since they are most easily maintained and manipulated. Unicellular screening systems also often use easily detectable changes in phenotype to monitor the effect of test compounds on the cell. Unicellular organisms, however, are inadequate models for estimating the potential effects of many compounds on complex multicellular animals, as they do not have the ability to carry out biotransformations to the extent or at levels found in higher organisms.

The biotransformation of chemical compounds by multicellular organisms is a significant factor in determining the effects, including toxicity, of agents to which they are exposed. Accordingly, multicellular screening systems may be preferred or required to detect the toxic effects of compounds. The use of multicellular organisms as screening tools has been significantly hampered, however, by the lack of convenient screening mechanisms or endpoints, such as those available in yeast or bacterial systems. In an attempt to compensate for the deficiencies of single cell testing systems, animal models using small laboratory species such as rats and mice have been developed. Such models, however, do not always provide an accurate picture of cellular responses induced in higher mammals such as humans. Accordingly, higher order mammals such as dogs are often required in the later stages of pharmaceutical testing or in testing the biological effects of known or potential toxins.

In addition, safety guidelines in the pharmaceutical, food and chemical industries in many countries require pre-clinical toxicity testing of every product in at least two species, one rodent species, usually the rat, and one non-rodent species, usually the dog (Smith et al., Lab Anim 35(2):117-130 (2001); Broadhead et al., Hum Exp Toxicol 19(8):440-447 (2000); Zbinden, Regul Toxicol Pharmacol 17(1):85-94 (1993)). accordance with legal requirements for acute and repeated-dose toxicity testing, large-scale studies are usually undertaken, entailing the use of many dogs. Although primates, such as macaques and marmosets, may also be used as the non-rodent, large animal species, it is likely that the dog will remain the principal large animal used in testing.

There have been recent attempts in the pharmaceutical industry to redesign pre-clinical testing, so that fewer animals can be used and so that their use is more targeted. Because toxicity data from testing in dogs is known to be predictive for humans, testing in dogs, however, cannot be eliminated.

Thus, there is a need for sensitive and rapid methods of detecting cellular responses and differential gene expression in animal models in response to therapeutic agents, particularly methods that can accommodate large numbers of samples. Techniques employing microarrays, especially microarrays containing a high percentage of a large animal's genome (such as a dog's) are, therefore, likely to be the most useful in providing information about responses to therapeutic agents or toxins that would be seen in other large animals, such as humans.

### SUMMARY OF THE INVENTION

The present invention includes a set of cDNA sequences representative of the expressed genome of a dog. The present invention also includes microarrays containing probes that hybridize to mRNA sequences corresponding to the canine genes. The sequences on these microarrays represent a large portion of the canine genome, and these microarrays are capable of detecting changes in gene expression level in a large percentage of canine genes.

Additionally, the present invention includes methods of using the microarray chips to detect or monitor changes in gene expression in a tissue or cell sample, such as a toxic response in dogs after exposure of the dogs to a known toxin or to a compound with unknown toxic properties. The microarray chips are capable of detecting up- or down-regulation of a large percentage of the genes in the canine genome following exposure of the animal to a known or unknown toxin, and a profile of the genes that are up- and/or down-regulated can be produced. Genes within the profile can be selected as marker genes and their expression level determined in subjects undergoing toxicity response testing. The methods of the present invention may also be used to detect genes that are up- or down-regulated in canines in a disease state. A profile of these genes may then be produced, and marker genes may be identified. Expression levels of these genes may be used in the identification and monitoring of diseases in canines. In addition, expression levels of genes identified as marker genes may be used to detect and monitor a positive or negative response to a medical or pharmaceutical treatment.

The present invention also includes a computer system comprising a database of the genes and gene fragments herein described, in which the database also includes information identifying the expression level of genes in at least one tissue or cell sample, such as normal and toxin-exposed canine tissues. The database may also include descriptive information from external databases. Further, the present invention includes methods of using the computer system to present information comparing the expression level of the genes in the database in normal and in toxin-exposed tissues and cells.

Finally, the present invention includes kits comprising the canine microarrays, along with sequence information and gene expression information regarding the gene expression levels in at least one tissue or cell sample.

### DETAILED DESCRIPTION

Many biological functions are accomplished by altering the expression of various genes through transcriptional (e.g. through control of initiation, provision of RNA precursors, RNA processing, etc.) and/or translational control. For example, fundamental biological processes such as cell cycle, cell differentiation and cell death are often characterized by the variations in the expression levels of groups of genes.

Changes in gene expression are also associated with the effects of various chemicals, drugs, toxins, pharmaceutical agents and pollutants on an organism or cells. For example, the lack of sufficient expression of functional tumor suppressor genes and/or the over expression of oncogene/protooncogenes after exposure to all agent could lead to tumorgenesis or hyperplastic growth of cells (Marshall, Cell, 64: 313-326 (1991); Weinberg, Science, 254:1138-1146 (1991)). Thus, changes in the expression levels of particular genes (e.g. oncogenes or tumor suppressors) may serve as signposts for the presence and progression of toxicity or other cellular responses to exposure to a particular compound.

Monitoring changes in gene expression may also provide certain advantages during drug screening and development. Often drugs are screened for the ability to interact with a major target without regard to other effects the drugs have on cells. These cellular effects may cause toxicity in the whole animal, which prevents the development and clinical use of the potential drug.

The present invention is based, in part, on the identification of new canine genes, including new canine genes that are expressed in one or more tissues, such as liver, kidney, heart, brain and testicular tissue. These genes correspond to the canine cDNA of SEQ ID NOS: 1-11,109.

The genes of the invention may be used as diagnostic agents or markers to detect a cellular response in a sample individually or as part of a gene expression profile. They can also serve as a target for agents that modulate gene expression or activity. For example, agents may be identified that modulate gene expression levels as a means of modulating aberrant biological processes associated with a cellular response, such as inflammation, cytotoxicity, hyperplastic growth or disruption of the cell cycle.

Nucleic Acid Molecules

The present invention provides nucleic acid molecules corresponding to the genes or sequences described herein, preferably in isolated form. As used herein, “nucleic acid” includes RNA or DNA that comprises any one of SEQ ID NOS:1-11,109, is complementary to any of these sequences, specifically hybridizes to a nucleic acid of SEQ ID NOS: 1-11,109 and remains stably bound to it under appropriate stringency conditions, and/or exhibits greater than about 90% or 95% or more nucleotide sequence identity through greater than about 90% or 95% of the sequence length of SEQ ID NOS: 1-11,109.

Specifically contemplated are genomic DNA, cDNA, mRNA and antisense molecules, as well as nucleic acids based on alternative backbones or including alternative bases, whether derived from natural sources or synthesized. Such hybridizing or complementary nucleic acids, however, are defined further as being novel and unobvious over any prior art nucleic acid including that which encodes, hybridizes under appropriate stringency conditions, or is complementary to nucleic acid encoding a protein according to the present invention.

Homology or identity at the nucleotide or amino acid sequence level is determined by BLAST (Basic Local Alignment Search Tool) analysis using the algorithm employed by the programs blastp, blastn, blastx, tblastn and tblastx (Altschul S. F. et al., Nucleic Acids Res 25:3389-3402 (1997), and Karlin et al., Proc Natl Acad Sci USA 87:2264-2268 (1990), both fully incorporated by reference) which are tailored for sequence similarity searching. The approach used by the BLAST program is to first consider similar segments, with and without gaps, between a query sequence and a database sequence, then to evaluate the statistical significance of all matches that are identified and finally to summarize only those matches which satisfy a pre-selected threshold of significance. For a discussion of basic issues in similarity searching of sequence databases, see Altschul et al., Nature Genetics 6:119-129 (1994), which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al., Proc Natl Acad Sci USA 89: 10915-10919 (1992), fully incorporated by reference), recommended for query sequences over 85 nucleotides or amino acids in length.

For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters were adjusted as follows: Q=10 (gap creation penalty); R=10 (gap extension penalty); E value=10 (expected number of matches in the sequence database(s) purely by chance based on a random sequence model; word size=11 The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” are those that (1) employ low ionic strength and high temperature for washing, for example, 0.015 M NaCl/0.0015 M sodium citrate/0.1% SDS at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 MM NaCl, 75 mM sodium citrate at 42° C. Another example is hybridization in 50% formamide, 5 SSC (0.75 M NaCl, 0.075 M sodium citrate), 50 mM sodium phosphate (pH 6.8), 0.1% sodium pyrophosphate, 5 Denhardt's solution, sonicated salmon sperm DNA (50 μg/ml), 0.1% SDS, and 10% dextran sulfate at 42° C., with washes at 42° C. in 0.2 SSC and 0.1% SDS. A skilled artisan can readily determine and vary the stringency conditions appropriately to obtain a clear and detectable hybridization signal. When hybridizing an oligonucleotide to MRNA or cRNA from a cell sample, the “stringent conditions” under which the oligonucleotide or probe specifically binds to a nucleic acid molecule of the invention can be calculated by one of ordinary skill in the art (see below).

As used herein, a nucleic acid molecule is said to be “isolated” when the nucleic acid molecule is substantially separated from contaminant nucleic acid molecules encoding other polypeptides.

The present invention further includes fragments of the nucleic acid molecules as herein described, e.g. hybridization probes or oligonucleotides. As used herein, a fragment of a nucleic acid molecule refers to a small portion of a sequence as herein described. The size of the fragment will be determined by the intended use. For example, if the fragment is chosen so as to encode a protein or an active portion of the protein, the fragment will need to be large enough to encode the full protein or the functional region(s) of the protein. For instance, fragments which encode peptides corresponding to predicted antigenic regions may be prepared. If the fragment is to be used as a nucleic acid probe or PCR primer, then the fragment length is chosen so as to obtain a relatively small number of false positives during probing/priming.

Fragments of the nucleic acid molecules of the present invention (i.e., synthetic oligonucleotides) that are used as probes or specific primers for the polymerase chain reaction (PCR), or to synthesize gene sequences encoding proteins, can easily be synthesized by chemical techniques, for example, the phosphoramidite method of Matteucci et al. (J Am Chem Soc 103:3185-3191 (1981)) or-using automated synthesis methods. In addition, larger DNA segments can readily be prepared by well known methods, such as synthesis of a group of oligonucleotides that define various modular segments of the gene, followed by ligation of oligonucleotides to build the complete modified gene.

The nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention.

rDNA Molecules Containing a Nucleic Acid Molecule

The present invention further provides recombinant DNA molecules (rDNAs) that comprise any one of SEQ ID NOS: 1-11,109. As used herein, a rDNA molecule is a DNA molecule that has been subjected to molecular manipulation in situ. Methods for generating rDNA molecules are well known in the art, for example, see Sambrook et al., Molecular Cloning—A Laboratory Manual, 3d Ed., Cold Spring Harbor Laboratory Press, Cold Spring Harbor, N.Y., 2001. In the preferred rDNA molecules, a DNA sequence is operably linked to replication or expression control sequences and/or vector sequences.

The choice of control sequences to which one of the sequences of the present invention is operably linked depends directly, as is well known in the art, on the functional properties desired, e.g., protein expression, replication requirements and the host cell to be transformed. A vector contemplated by the present invention is at least capable of directing the replication or insertion into the host chromosome, and, in certain cases, expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restrict ion sites for insertion of a DNA segment of the present invention. Typical of such vector plasmids are pUC8, pUC9, pBR322 and pBR329 available from BioRad Laboratories, (Richmond, Calif.), pPL and pKK223 available from Pharmacia (Piscataway, N.J.).

Expression vectors compatible with eukaryotic cells, preferably those compatible with vertebrate cells, such as canine cells, can also be used to form rDNA molecules that contain a coding sequence. Eukaryotic cell expression vectors, including viral vectors, are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d. (International Biotechnologies, Inc.), pTDT1 (ATCC, #31255), the vector pCDM8 described herein, and the like eukaryotic expression vectors. Vectors may be modified to include prostate cell specific promoters if needed.

Eukaryotic cell vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al., J Mol Anal Genet 1:327-341 (1982)) Alternatively, the selectable marker can be present on a separate plasmid, and the two vectors are introduced by co-transfection of the host cell, and selected by culturing in the appropriate drug for the selectable marker.

Host Cells Containing an Exogenously Supplied Coding Nucleic Acid Molecule

The present invention further provides host cells transformed with a nucleic acid molecule of the present invention. The host cell can be either prokaryotic or eukaryotic. Eukaryotic cells useful for expression of proteins are not limited, so long as the cell line is compatible with cell culture methods and compatible with the propagation of the expression vector and possible expression of the gene product. Preferred eukaryotic host cells include, but are not limited to, yeast, insect and mammalian cells, preferably vertebrate cells such as those from a mouse, rat, monkey, human or canine cell line. Preferred eukaryotic host cells include Chinese hamster ovary (CHO) cells available from the ATCC as CCL61, NIH Swiss mouse embryo cells (NIH/3T3) available from the ATCC as CRL 1658, baby hamster kidney cells (BHK), and the like eukaryotic tissue culture cell lines.

Any prokaryotic host can be used to replicate a rDNA molecule of the invention. The preferred prokaryotic host is E. coli.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Cohen et al., (1972) Proc Natl Acad Sci USA 69:2110 (1972); and Sambrook et al. (supra). With regard to transformation of vertebrate cells with vectors containing rDNAs, electroporation, cationic lipid or salt treatment methods are typically employed, see, for example, Graham et al., Virol 52:456 (1973); and Wigler et al., Proc Natl Acad Sci USA 76:1373-1376 (1979).

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, J Mol Biol 98:503 (1975) or Berent et al., Biotech 3:208 (1985), or the proteins produced from the cell assayed via an immunological method.

Nucleic Acid Assay Formats

The genes and sequences described herein may be used in a variety of nucleic acid detection assays to detect or quantititate the expression level of a gene or multiple genes in a given sample.

Any assay format to detect gene expression may be used. For example, traditional Northern blotting, dot or slot blot, nuclease protection, primer directed amplification, RT-PCR, semi- or quantitative PCR, branched-chain DNA and differential display methods may be used for detecting gene expression levels. Those methods are useful for some embodiments of the invention. In cases where smaller numbers of genes are detected, amplification based assays may be most efficient. Methods and assays of the invention, however, may be most efficiently designed with hybridization-based methods for detecting the expression of a large number of genes.

Any hybridization assay format may be used, including solution-based and solid support-based assay formats. Solid supports containing oligonucleotide probes based on the genes of the invention can be filters, polyvinyl chloride dishes, particles, beads, microparticles or silicon or glass based chips, etc. Such chips, wafers and hybridization methods are widely available, for example, those disclosed by Beattie (WO 95/11755).

Any solid surface to which oligonucleotides can be bound, either directly or indirectly, either covalently or non-covalently, can be used. A preferred solid support is a high density array or DNA chip. Solid supports also include beads, sets of beads, membranes, and other formats using any material, including glass and/or silicon. When beads or sets of beads are the support, one or more than one species of probe or oligonucleotide may be attached to each bead. In one embodiment, each species of probe or oligonucleotide is attached each to a different bead, and the set of beads comprises all or a subset of the nucleic acid molecules described herein. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 or more of such features on a single solid support. The solid support, or the area within which the probes are attached may be on the order of about a square centimeter. For instance, about 10,000, 100,000 or more probes may be attached per square centimeter. Probes may be attached to single or multiple solid support structures, e.g., the probes may be attached to a single chip or to multiple chips to comprise a chip set.

Oligonucleotide probe arrays for expression monitoring can be made and used according to any techniques known in the art (see for example, Lockhart et al., Nat Biotechnol 14:1675-1680 (1996); McGall et al., Proc Nat Acad Sci USA 93:13555-13460 (1996)). Such probe arrays may contain at least one or more oligonucleotides that are complementary to or hybridize to one or more of the genes or their transcripts. For instance, such arrays may contain oligonucleotides that are complementary or hybridize to at least 1, 2, 3, 4, 5, 6, 7, 8, 9, 10, 20, 30, 50, 70, 100, 500, 1000, 2000, 5000, 10,000 or more of the genes described herein. Preferred arrays contain all or nearly all of the genes described herein, for instance, at least about 90%, 95%, 97%, 99% or 99.5% of the sequences herein described. In a preferred embodiment, arrays are constructed that contain oligonucleotides to detect all or nearly all of the genes on a solid support substrate, such as a chip. Such arrays may represent all or nearly all of the entire expressed genome of a dog.

As described above, in addition to the sequences disclosed, sequences such as naturally occurring variant or polymorphic sequences may be used in the methods and compositions of the invention. For instance, expression levels of various allelic or homologous forms of a gene may be assayed. Any and all nucleotide variations that do not alter the functional activity of a gene, including all naturally occurring allelic variants of the genes herein disclosed, may be used in the methods and to make the compositions (e.g., arrays) of the invention.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes refer to oligonucleotides that are capable of hybridizing under stringent conditions to at least part of the nucleotide sequences of said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g. the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. One-of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” refers to the binding, duplexing, or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, about 1000, about 10,000 or about 1,000,000 different nucleic acid hybridizations.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

The terms “mismatch control” or “mismatch probe” refer to a probe whose sequence is deliberately selected not to be perfectly complementary to a particular target sequence. For each mismatch CAM) control in a high-density array there typically exists a corresponding perfect match (PM) probe that is perfectly complementary to the same particular target sequence. The mismatch may comprise one or more bases.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical submit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights. In an embodiment of the invention, the percent sequence identity is at least about 90% across 90% of the entire length of a given sequence.

Probe Design

One of skill in the art will appreciate that an enormous number of array designs are suitable for the practice of this invention. The high density array will typically include a number of test probes that specifically hybridize to the sequences of interest. Probes may be produced from any region of the genes identified herein and the attached representative sequence listing. See WO 99/32660 for methods of producing probes for a given gene or genes. In addition, any available software may be used to produce specific probe sequences, including, for instance, software available from Molecular Biology Insights, Olympus Optical Co. and Premier Biosoft International. In a preferred embodiment, the array will also include one or more control probes.

High density array chips of the invention include “test probes.” Test probes may be oligonucleotides that range from about 5 to about 500, or about 7 to about 50 nucleotides, more preferably from about 10 to about 40 nucleotides and most preferably from about 15 to about 35 nucleotides in length. In other particularly preferred embodiments, the probes are 20 or 25 nucleotides in length. In another preferred embodiment, test probes are double or single strand DNA sequences. DNA sequences are isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes may fall into three categories referred to herein as 1) normalization controls; 2) expression level controls; and 3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent) Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present the perfect match probes should be consistently brighter than the mismatch probes. In addition, if all central mismatches are present, the mismatch probes can be used to detect a mutation, for instance, a mutation of a gene comprising one of SEQ ID NOS: 1-11,109. The difference in intensity between the perfect match and the mismatch probe provides a good measure of the concentration of the hybridized material.

Nucleic Acid Samples

Any canine cell or tissue sample may be used in the methods and assays of the invention. Cell or tissue samples used in the assays of the invention may be produced, grown, cultured, etc. in vitro or in vivo. When cultured cells or tissues are used, appropriate mammalian liver extracts may also be added with a test agent to evaluate agents that may require biotransformation to exhibit toxicity. In a preferred format, primary isolates of animal or canine hepatocytes which already express the appropriate complement of drug-metabolizing enzymes may be exposed to the test agent without the addition of mammalian liver extracts.

The genes which are assayed according to the present invention are typically in the form of mRNA or reverse transcribed mRNA. The genes may be cloned or not. The genes may be amplified or not. The cloning and/or amplification do not appear to bias the representation of genes within a population. In some assays, it may be preferable, however, to use polyA+RNA as a source, as it can be used with less processing steps.

As is apparent to one of ordinary skill in the art, nucleic acid samples used in the methods and assays of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology, Vol. 24, Hybridization With Nucleic Acid Probes: Theory and Nucleic Acid Probes, P. Tijssen, Ed., Elsevier Press, New York, 1993. Such samples include RNA samples, but also include cDNA synthesized from a mRNA sample isolated from a cell or tissue of interest. Such samples also include DNA amplified from the cDNA, and RNA transcribed from the amplified DNA. One of skill in the art would appreciate that it is desirable to inhibit or destroy RNase present in homogenates before homogenates are used.

Biological samples may be of any biological tissue or fluid or cells, as well as cells raised in vitro, such as cell lines and tissue culture cells. Frequently the sample will be a tissue or cell sample that has been exposed to a compound, agent, drug, pharmaceutical composition, potential environmental pollutant or other composition. In some formats, the sample will be a “clinical sample.” Typical clinical samples include, but are not limited to, blood, blood-cells (e.g., white cells), tissue or fine needle biopsy samples, urine, peritoneal fluid, and pleural fluid, or cells therefrom.

Biological samples may also include sections of tissues, such as frozen sections or formalin fixed sections taken for histological purposes.

Forming High Density Arrays

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency, in this case in 6× SSPET at 37° C. (0.005% Triton X-100), to ensure hybridization and then subsequent washes are performed at higher stringency (e.g., 1× SSPET at 37° C.) to eliminate mismatched hybrid duplexes. Successive washes may be performed at increasingly higher stringency (e.g., down to as low as 0.25× SSPET at 37° C. to 50° C.) until a desired level of hybridization specificity is obtained. Stringency can also be increased by addition of agents such as formamide. Hybridization specificity may be evaluated by comparison of hybridization to the test-probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art. See WO 99/32660.

Databases

The present invention includes relational databases containing sequence information, for instance, for the genes herein described, as well as gene expression information from tissue or cells, such as canine cells or tissue exposed to various standard compounds, such as toxins. Databases may also contain information associated with a given sequence or tissue sample such as descriptive information about the gene associated with the sequence information, or descriptive information concerning the clinical status of the tissue sample, or the animal from which the sample was derived. The database may be designed to include different parts, for instance a sequence database and a gene expression database. Methods for the configuration and construction of such databases and computer-readable media to which such databases are saved are widely available, for instance, see U.S. Pat. No. 5,953,727, which is herein incorporated by reference in its entirety.

The databases of the invention may be linked to an outside or external database such as GenBank (www.ncbi.nlm.nih.gov/entrez.index.html); KEGG (www.genome.ad.jp/kegg); SPAD (www.grt.kyzushu-u.ac.jp/spad/index.html); HUGO (www.gene.ucl.ac.uk/hugo); Swiss-Prot (www.expasy.ch.sprot); Prosite (www.expasy.ch/tools/scnpsit1.html); OMIM (www.ncbi.nlm.nih.gov/omin); and GDB (www.gdb.org). In a preferred embodiment, the external database is GenBank and the associated databases maintained by the National Center for Biotechnology Information (NCBI) (www.ncbi.nlm.nih.gov).

Any appropriate computer platform, user interface, etc. may be used to perform the necessary comparisons between sequence information, gene expression information and any other information in the database or information provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, electronic Northerns that allow the user to determine the cell type or tissue in which a given gene is expressed and to allow determination of the abundance or expression level of a given gene in a particular tissue or cell.

The databases of the invention may also be used to present information identifying the expression level in a tissue or cell of a set of genes comprising one or more of the genes of SEQ ID NOS: 1-11,109, comprising the step of comparing the expression level of at least one gene in a cell or tissue exposed to a test agent to the level of expression of the gene in the database. Such methods may be used to predict the toxic potential of a given compound by comparing the level of expression of a gene or genes from a tissue or cell sample exposed to the test agent to the expression levels found in a control tissue or cell samples exposed to a standard toxin or hepatotoxin such as those herein described. Such methods may also be used in the drug or agent screening assays as described herein.

Kits

The invention further includes kits combining, in different combinations, high-density oligonucleotide arrays, reagents for use with the arrays, protein reagents encoded by the genes herein described, signal detection and array-processing instruments, gene expression databases and analysis and database management software described above. The kits may be used, for example, to predict or model the toxic response of a test compound, to monitor the progression of disease states, to identify genes that show promise as new drug targets and to screen known and newly designed drugs as discussed above.

The databases packaged with the kits may be a compilation of expression patterns of the genes in various tissues or in tissues, including cell or tissue samples, exposed to various compounds or reference toxins. In particular, the database software and packaged information that may contain the databases saved to a computer-readable medium include the expression results of the genes that can be used to predict toxicity of a test agent, by comparing the expression levels of the genes induced by the test agent to the expression levels in control samples. In another format, database and software information may be provided in a remote electronic format, such as a website, the address of which may be packaged in the kit.

The kits may used in the pharmaceutical industry, where the need for early drug testing is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Databases and software designed for use with use with microarrays is discussed in Balaban et al., U.S. Pat. No. 6,229,911, a computer-implemented method for managing information, stored as indexed tables, collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164,1 discloses a software-based method for identifying mutations in a nucleic acid sequence based on differences in probe fluorescence intensities between wild type and mutant sequences that hybridize to reference sequences.

Identification of Marker Genes

Cell or tissue samples such as those associated with a disease state, for example, may be analyzed using the microarray chip of the invention, and gene expression profiles may be prepared. Expression levels of genes identified as marker genes, based on their properties as an indicator of a disease state, or as an indicator of normal functioning, for example, may be measured and then used to monitor a variety of medical treatments or in diagnostic procedures. Marker genes may be used in pharmaceutical development to monitor the degree of apoptosis or effect of treatment with pharmaceuticals, such as beta-adrenergic blocking agents. Additionally, the expression level of genes involved in the development of carcinomas or autoimmune disorders may be measured. In gene therapy, monitoring the expression of marker genes provides an indication of the level of genes delivered by various viral and synthetic non-viral vectors.

Identification of Toxicity Markers

To evaluate and identify gene expression changes that are predictive of toxicity, studies using selected compounds with well characterized toxicity can be used to catalogue altered gene expression during exposure in vivo and in vitro. For instance, canine cell or tissue samples can be prepared by administering a toxin or a control to a canine subject and harvesting tissue or cell samples after exposure. In another embodiment, in vitro cultured canine cells are exposed to the toxin. Methods of exposure or administration and methods of preparing cell or tissue samples are well known in the art. See, for example, PCT publication nos. WO 02/10453 and WO 02/095000, as well as PCT application nos. PCT/US02/21735, filed Jul. 10, 2002, and PCT/US03/03194, filed Jan. 31, 2003, all of which are herein incorporated by reference. In the instant invention, standard known toxins such as acyclovir, amitryptiline, alpha-naphthylisothiocyante (ANIT), acetaminophen, AY-25329, bicalutamide, carbon tetrachloride, chloroform, clofibrate, cyproterone acetate (CPA), diclofenac, diflunisal, dioxin, 17α-ethinylestradiol, hydrazine, indomethacin, lipopolysaccharide, phenobarbital, tacrine, valproate, WY-14643, zileuton, methotrexate, lovastatin, mercuric chloride, cephaloridine, ifosfamide, cyclophosphamide and minoxidil, 2-acetylaminofluorene (2-AAF), amiodarone, BI liver toxin, carbamazepine, chlorpromazine, CI-1000, colchicine, dimethylnitrosamine (DMN), gemfibrozil, imipramine, menadione, tamoxifen, tetracycline, thioacetamide, adriamycin, bromoethylamine HBr, carboplatin, cidorfovir, cis-platin, citrinin, cyclophosphamide, gentamicin, hydralizine, lithium, pamindronate, puromycin aminonucleoside, sulfadiazine, sodium chromate, sodium oxalate, vancomycin, BI-QT, clenbuterol, isoproteranol, norepinephrine, epinephrine, amphotericin B, epirubicin, phenylpropanolamine, rosiglitazone and 1-methyl-4phenyl-1,2,3,6-tetrahydropyridine HCl (MPTP) may be used to produce toxin-specific and composite gene expression profiles.

Toxicity Prediction and Modeling

The genes and gene expression information, as well as the portfolios and subsets of the genes that may be identified using the sequences and arrays of the invention, may be used to predict at least one toxic effect, such as the hepatotoxicity or nephrotoxicity of a test or unknown compound. As used, herein, at least one toxic effect includes, but is not limited to, a detrimental change in the physiological status of a cell or organism. The response may be, but is not required to be, associated with a particular pathology, such as tissue necrosis. The response may be associated with all or only part of an organ, e.g., renal tubular necrosis or glomerulonephritis. Additionally, the toxic effect includes effects at the molecular and cellular level. Hepatotoxicity is an effect as used herein and includes but is not limited to the pathologies of liver necrosis, hepatitis, fatty liver and protein adduct formation.

In general, assays to predict the toxicity of a test agent (or compound or multi-component composition) comprise the steps of exposing a cell population to the test compound, assaying or measuring the level of relative or absolute gene expression of one or more of the genes as herein described and comparing the identified expression level(s) to the expression level(s) found for a standard toxin. Assays may include the measurement of the expression levels of about 1, 2, 3, 4, 5, 6, 7, 8, 9, 10, 15, 20, 25, 30, 50, 75, 100, 500, 1000, 5000, 10,000 or more genes.

In the methods of the invention, the gene expression level for a gene or genes induced by the test agent, compound or compositions may be comparable to the levels found in the databases disclosed herein, or in other samples, such as toxin-exposed samples, if the expression level varies within a factor of about 2, about 1.5 or about 1.0 fold. In some cases, the expression levels are comparable if the agent induces a change in the expression of a gene in the same direction (e.g., up or down) as a reference toxin.

The cell population that is exposed to the test agent, compound or composition may be exposed in vitro or in vivo. For instance, cultured or freshly isolated hepatocytes, in particular dog hepatocytes, may be exposed to the agent under standard laboratory and cell culture conditions. In another assay format, in vivo exposure may be accomplished by administration of the agent to a living animal, for instance a laboratory dog.

Procedures for designing and conducting toxicity tests in in vitro and in vivo systems are well known, and are described in many texts on the subject, such as Loomis et al., Loomis's Esstentials of Toxicology 4th Ed., Academic Press, New York, 1996; Echobichon, The Basics of Toxicity Testing, CRC Press, Boca Raton, 1992; Frazier, editor, In Vitro Toxicity Testing, Marcel Dekker, New York, 1992; and the like.

In in vitro toxicity testing, two groups of test organisms are usually employed: One group serves as a control and the other group receives the test compound in a single dose (for acute toxicity tests) or a regimen of doses (for prolonged or chronic toxicity tests). Because, in some cases, the extraction of tissue as called for in the methods of the invention requires sacrificing the test animal, both the control group and the group receiving compound must be large enough to permit removal of animals for sampling tissues, if it is desired to observe the dynamics of gene expression through the duration of an experiment.

In setting up a toxicity study, extensive guidance is provided in the literature for selecting the appropriate test organism for the compound being tested, route of administration. dose ranges, and the like. Water or physiological saline (0.9% NaCl in water) is the solute of choice for the test compound since these solvents permit administration by a variety of routes. When this is not possible because of solubility limitations, vegetable oils such as corn oil or organic solvents such as propylene glycol may be used.

Regardless of the route of administration, the volume required to administer a given dose is limited by the size of the animal that is used. It is desirable to keep the volume of each dose uniform within and between groups of animals. Even when aqueous or physiological saline solutions are used for parenteral injection, the volumes that are tolerated are limited, although such solutions are ordinarily thought of as being innocuous. In some instances, the route of administration to the test animal should be the same as, or as similar as possible to, the route of administration of the compound to man for therapeutic purposes.

When a compound is to be administered by inhalation, special techniques for generating test atmospheres are necessary. The methods usually involve aerosolization or nebulization of fluids containing the compound. If the agent to be tested is a fluid that has an appreciable vapor pressure, it may be administered by passing air through the solution under controlled temperature conditions. Under these conditions, dose is estimated from the volume of air inhaled per unit time, the temperature of the solution, and the vapor pressure of the agent involved. Gases are metered from reservoirs. When particles of a solution are to be administered, unless the particle size is less than about 2 μm the particles will not reach the terminal alveolar sacs in the lungs. A variety of apparatuses and chambers are available to perform studies for detecting effects of irritant or other toxic endpoints when they are administered by inhalation. The preferred method of administering an agent to animals is via the oral route, either by intubation or by incorporating the agent in the feed.

When the agent is exposed to cells in vitro or in cell culture, the cell population to be exposed to the agent may be divided into two or more subpopulations, for instance, by dividing the population into two or more identical aliquots. In some preferred embodiments of the methods of the invention, the cells to be exposed to the agent are derived from liver tissue. For instance, cultured or freshly isolated rat hepatocytes may be used.

The methods of the invention may be used to generally predict at least one toxic response, and as described in the Examples, may be used to predict the likelihood that a compound or test agent will induce various specific pathologies such as those of the liver (liver necrosis, fatty liver disease, protein adduct formation or hepatitis), those of the kidney, heart, brain or testes, or other pathologies associated with at least one of the toxins herein described. The methods of the invention may also be used to determine the similarity of a toxic response to one or more individual compounds. In addition, the methods of the invention may be used to predict or elucidate the potential cellular pathways influenced, induced or modulated by the compound or test agent due to the similarity of the expression profile compared to the profile induced by a known toxin.

Diagnostic Uses for the Toxicity Markers

As described above, the genes and gene expression information or portfolios of the genes with their expression information may be used as diagnostic markers for the prediction or identification of the physiological state of tissue or cell sample that has been exposed to a compound or to identify or predict the toxic effects of a compound or agent. For instance, a tissue sample such as a sample of peripheral blood cells or some other easily obtainable tissue sample may be assayed by any of the methods described above, and the expression levels from a gene or genes may be compared to the expression levels found in tissues or cells exposed to the toxins described herein. These methods may result in the diagnosis of a physiological state in the cell or may be used to identify the potential toxicity of a compound, for instance a new or unknown compound or agent. The comparison of expression data, as well as available sequence or other information may be done by researcher or diagnostician or may be done with the aid of a computer and databases as described below.

In another format, the levels of a gene or genes, the encoded protein(s), or any metabolite produced by the encoded protein may be monitored or detected in a sample, such as a bodily tissue or fluid sample to identify or diagnose a physiological state of an organism. Such samples may include any tissue or fluid sample, including urine, blood and easily obtainable cells such as peripheral lymphocytes.

Use of the Markers for Monitoring Toxicity Progression

As described above, the genes and gene expression information provided may also be used as markers for the monitoring of toxicity progression, such as that found after initial exposure to a drug, drug candidate, toxin, pollutant, etc. For instance, a tissue or cell sample may be assayed by any of the methods described above, and the expression levels from a gene or genes may be compared to the expression levels found in tissue or cells exposed to a standard toxin or toxins. The comparison of the expression data, as well as available sequence or other information may be done by researcher or diagnostician or may be done with the aid of a computer and databases.

Use of the Toxicity Markers for Drug Screening

According to the present invention, the genes and arrays described herein may be used to identify markers or drug targets to evaluate the effects of a candidate drug, chemical compound or other agent on a cell or tissue sample. For instance, the genes may also be used as drug targets to screen for agents that modulate their expression and/or activity. In various formats, a candidate drug or agent can be screened for the ability to simulate the transcription or expression of a given marker or markers or to down-regulate or counteract the transcription or expression of a marker or markers. According to the present invention, one can also compare the specificity of a drug's effects by looking at the number of markers which the drug induces and comparing them. More specific drugs will have less transcriptional targets. Similar sets of markers identified for two drugs may indicate a similarity of effects.

Assays to monitor the expression of a marker or markers may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to one, two or more genes as described herein may be used to directly monitor or detect changes in gene expression in the treated or exposed cell. Cell lines, tissues or other samples are first exposed to a test agent and in some instances, a known toxin, and the detected expression levels of one or more, or preferably 2 or more of the genes are compared to the expression levels of those same genes exposed to a known toxin alone. Compounds that modulate the expression patterns of the known toxin(s) would be expected to modulate potential toxic physiological effects in vivo.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see G. A. Grant in: Molecular Biology and Biotechnology, Meyers, ed., pp. 659-664, VCH Publishers, New York, 1995). A skilled artisan can readily recognize that there is no limit as to the structural nature of the agents of the present invention.

Use of Assays and Genes for Veterinary Medicine

The genes and arrays described herein may be used in veterinary medicine, for instance, to produce canine gene expression profiles indicative of a disease or physiological state. For instance, gene expression profiles may be created using arrays of the invention from peripheral blood cells isolated from an animal with a known disease state, for example, an inflammatory disease. Such gene expression profiles can then be used as diagnostic or therapeutic markers to aid in prediction of disease, to monitor treatment progression or efficacy, or to monitor disease progression (see WO 99/10536).

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Identification of Canine Nucleic Acid Sequences

A cDNA library of mixed canine tissues (liver, kidney, heart, brain and testes) was produced according to standard methods. Following 3′EST sequencing to identify individually expressed genes and gene fragments, these genes and gene fragments were further sequenced and were analyzed for their homology to known sequences. Only sequences that showed alignment below a first threshold level (90%) to sequences in public databases and that had identity below a second threshold level (90%) within the region of alignment were used to prepare microarrays.

### Example 2

### Preparation of Canine Microarray

Oligonucleotides of approximately 25 bases, corresponding to various regions of the novel genes identified above, were synthesized according to standard methods. The oligonucleotides were spotted onto microchips according to the Affymetrix photolithography protocol to create microarrays with over 100,000 sequences per chip. The chips were tested for intra-lot variability, inter-lot variability and day-to-day variability. The chips were also tested for the specificity of binding to canine RNA in hybridization experiments with RNA samples from various species: dog, human, rat and mouse. As sample preparation for testing the microarrays, total RNA was extracted from the following canine tissues and pooled: liver, kidney, heart, brain and testes. The pooled RNA was reverse transcribed to prepare cDNA and amplified by reaction with a reverse polymerase to prepare cRNA.

A samples from dogs hybridized to the chips to a considerably greater degree than samples from other species. The percentages of sequences on the chips that did and did not bind to RNA samples from other species are indicated in the following table.

| | ave. % | ave. % | ave. % | % genes at or above |
|---|---|---|---|---|
| organism | present | absent | marginal | 0.5 pM spike-in level |
| human | 7.1 | 91.2 | 1.8 | 3.5 |
| rat | 4.2 | 94.6 | 1.2 | 4.6 |
| mouse | 4.4 | 94.1 | 1.5 | 2.1 |

As a further control of specific hybridization, bacterial spikes were performed. Oligonucleotides designed from bacterial DNA sequences (Affymetrix) were incorporated into the microarrays, and canine RNA samples were spiked with known quantities of purified bacterial DNA (Affymetrix).

### Example 3

### Identification of Toxicity Markers and Toxicity Expression Profiles

Laboratory dogs are exposed to toxins, such as gentamicin, according to the following protocol. Gentamicin or vehicle (saline) is administered to dogs as shown below. The toxin is also prepared in saline solution.

| | | Dose Level | No. of | |
|---|---|---|---|---|
| Group | Drug | (mg/kg) | Males | Sacrifice |
| 1 | Saline | vehicle control | 5 | 6 hours after dosing |
| 2 | Gentamicin | X* | 5 | 6 hours after dosing |
| 3 | Gentamicin | Y* | 5 | 6 hours after dosing |
| 4 | Saline | vehicle control | 5 | 24 hours after dosing |
| 5 | Gentamicin | X | 5 | 24 hours after dosing |
| 6 | Gentamicin | Y | 5 | 24 hours after dosing |
| 7 | Saline | vehicle control | 5 | Day 7 |
| 8 | Gentamicin | X | 5 | Day 7 |
| 9 | Gentamicin | Y | 5 | Day 7 |

*X represents a safe but efficacious dose; Y represents a toxic or maximum-tolerated dose.

The toxin is administered daily by intramuscular injection. Animals were not dosed on the day of necropsy, with the exception of the 6-hour time point animals. ˜0.5 mL of blood from each animal is collected into an EDTA tube for analysis of plasma drug levels. Plasma (˜200 L) is obtained, frozen at ˜80° and used for test compound/metabolite estimation.

Animals are observed twice daily for signs of illness and drug toxicity (e.g., tremors, convulsions, salivation, diarrhea, lethargy, coma or other atypical behavior or appearance). were recorded as they occurred and included a time of onset, degree, and duration.

Blood samples are collected from each animal as follows. Approximately 1 mL of blood is collected into and EDTA tube for evaluation of hematology parameters. Approximately 1 mL of blood is collected into serum separator tubes for clinical chemistry analysis. An additional ˜2 mL of blood is collected into a 15 mL conical polypropylene vial to which ˜3 mL of Trizol is immediately added. The contents are mixed immediately with a vortex and by repeated inversion. The tubes are frozen in liquid nitrogen and stored at ˜−80° C.

At sacrifice, approximately 6 and 24 hours and 7 days after dosing, dogs scheduled for sacrifice are weighed, physically examined, and sacrificed by standard procedures using sterile, disposable instruments.

Fresh and sterile disposable instruments are used to collect tissues, with the exception of bone cutters that are-used to open the skull cap. These are sterilized between uses. All tissues are collected and frozen within approximately 5 minutes of the animal's death. The liver sections are frozen within approximately 2 minutes of the animal's death. The time of euthanasia, an interim time point at freezing of liver sections, and time at completion of necropsy are recorded. Tissues were stored at approximately −80° C., stored in liquid nitrogen, or preserved in 10% neutral buffered formalin.

Tissue collection is performed as follows. For the liver, the right medial lobe is snap frozen in liquid nitrogen and stored at ˜−80° C. The left medial lobe is preserved in 10% neutral-buffered formalin (NBF), and the left lateral lobe is snap frozen in liquid nitrogen and stored at ˜−80° C.

For the heart, a sagittal cross-section containing portions of the two atria and the two ventricles is preserved in 10% NBF for microscopic examination. The remaining heart is frozen in liquid nitrogen and stored at ˜−80° C.

For the kidneys, each kidney is hemi-dissected. Half is preserved in 10% NBF for microscopic examination, and the remaining half is frozen in liquid nitrogen and stored at ˜−80° C.

For the testes, a sagittal cross-section of each testis is preserved in 10% NBF for microscopic examination. The remaining testes are frozen together in liquid nitrogen and stored at ˜−80° C.

For the brain, a cross-section of the cerebral hemispheres and of the diencephalon is preserved in 10% NBF and the rest of the brain is frozen in liquid nitrogen and stored at ˜−80° C.

Microarray sample preparation is conducted with minor modifications, following the protocols set forth in the Affymetrix GeneChip Expression Analysis Manual. Frozen tissue is ground to a powder using a Spex Certiprep 6800 Freezer Mill. Total RNA is extracted with Trizol (GibcoBRL) utilizing the manufacturer's protocol. mRNA is isolated using the Oligotex mRNA Midi kit (Qiagen) followed by ethanol precipitation. Double stranded cDNA is generated from mRNA using the SuperScript Choice system (GibcoBRL). First strand cDNA synthesis is primed with a T7-(dT24) oligonucleotide. The cDNA is phenol-chloroform extracted and ethanol precipitated to a final concentration of 1 g/ml. From 2 g of cDNA, cRNA is synthesized using Ambion's T7 MegaScript in vitro Transcription Kit.

To biotin label the cRNA, nucleotides Bio-11-CTP and Bio-16-UTP (Enzo Diagnostics) are added to the reaction. Following a 37° C. incubation for six hours, impurities are removed from the labeled cRNA following the RNeasy Mini kit protocol (Qiagen). cRNA is fragmented (fragmentation buffer consisting of 200 mM Tris-acetate, pH 8.1, 500 mM KOAc, 150 mM MgOAc) for thirty-five minutes at 94° C. Following the Affymetrix protocol, 55 g of fragmented cRNA is hybridized on the array chip, or chip set, of the invention for twenty-four hours at 60 rpm in a 45° C. hybridization oven. The chips are washed and stained with Streptavidin Phycoerythrin (SAPE) (Molecular Probes) in Affymetrix fluidics stations. To amplify staining, SAPE solution is added twice, with an anti-streptavidin biotinylated antibody (Vector Laboratories) staining step in between. Hybridization to the probe arrays is detected by fluorometric scanning (Hewlett Packard Gene Array Scanner). Data is analyzed using Affymetrix GeneChip® version 3.0 and Expression Data Mining (EDMT) software (version 1.0), GeneExpress2000, and S-Plus.

Those genes that are differentially expressed upon exposure to gentamicin are identified using the microarray hybridization techniques described above, with data analysis according to a statistical method such as ANOVA, LDA or PCA (see WO 02/10453 or WO 02/095000). The set of genes that are differentially expressed creates an expression profile for a particular toxin. The determination of a particular gene expression profile in a tissue sample from a particular animal indicates a toxic response in that animal.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

1. An isolated nucleic acid molecule comprising any one of SEQ ID NOS: 1-11,109, the complement thereof, or a sequence exhibiting greater than 90% sequence identity across greater than 90% of the length of any one of SEQ ID NOS: 1-11,109.

2. A set of probes, wherein each of the probes comprises a sequence that specifically hybridizes to a gene or the transcript of a gene comprising any one of SEQ ID NOS: 1-11,109.

3. A set of probes according to claim 2, wherein the set comprises probes that specifically hybridize to at least 2 of the genes of SEQ ID NOS: 1-11,109.

4. A set of probes according to claim 2, wherein the set comprises probes that specifically hybridize to at least about 5 of the genes of SEQ ID NOS: 1-11,109.

5. A set of probes according to claim 2, wherein the set comprises probes that specifically hybridize to at least about 10 of the genes of SEQ ID NOS: 1-11,109.

6. A set of probes according to claim 2, wherein the set comprises probes that specifically hybridize to at least about 100 of the genes of SEQ ID NOS: 1-11,109.

7. A set of probes according to claim 2, wherein the set comprises probes that specifically hybridize to at least about 1000 of the genes of SEQ ID NOS: 1-11,109.

8. A set of probes according to claim 2, wherein the set comprises probes that specifically hybridize to about 99% of the genes of SEQ ID NOS: 1-11,109.

9. A set of probes according to claim 2, wherein the set comprises probes that specifically hybridize to all of the genes of SEQ ID NOS: 1-11,109.

10. A set of probes according to claim 2, wherein the probes are attached to a solid support.

11. A set of probes according to claim 10, wherein the solid support is selected from the group consisting of a membrane, a set of beads, a glass support and a silicon support.

12. A solid support comprising at least one probe, wherein each probe comprises a sequence that specifically hybridizes to a gene or the transcript of a gene comprising any one of SEQ ID NOS: 1-11,109.

13. A solid support of claim 12, wherein the solid support is an array comprising at least 10 different oligonucleotides in discrete locations per square centimeter.

14. A solid support of claim 12, wherein the array comprises at least 100 different oligonucleotides in discrete locations per square centimeter.

15. A solid support of claim 12, wherein the array comprises at least 1000 different oligonucleotides in discrete locations per square centimeter.

16. A solid support of claim 12, wherein the array comprises at least 10,000 different oligonucleotides in discrete locations per square centimeter.

17. A method of identifying tissue or cell markers, comprising:
(a) detecting the level of expression in a tissue or cell sample from a canine of one or more genes comprising SEQ ID NOS: 1-11,109; wherein differential expression of the one or more genes identifies a marker.

18. A method of claim 17, further comprising:
(b) comparing the level of expression of said one or more genes in step (a) to the level of expression of said genes in a control tissue or cell sample.

19. A method of claim 17, wherein the level of expression of one or more genes is detected with a probe that specifically hybridizes to a gene or a transcript of the gene.

20. A method of claim 19, wherein the probe is an oligonucleotide.

21. A method of claim 20, wherein the oligonucleotide is attached to a solid support.

22. A method of claim 21, wherein the solid support is a chip.

23. A method of claim 17, wherein the level of expression of one or more genes in step (a) is detected by polymerase chain amplification (PCR).

24. A method of claim 23, wherein the PCR is quantitative or semi-quantitative.

25. A method of claim 17, wherein step (a) comprises preparing cDNA from polyA-RNA isolated from the tissue or cell sample exposed to the toxin.

26. A method of claim 25, wherein cRNA is prepared from the cDNA.

27. A method of claim 17, wherein the tissue or cell sample is isolated from a dog or canine cells that have been exposed to a toxin.

28. A method of claim 17, wherein the tissue or cell sample is in vitro cultured.

29. A method of identifying toxicity markers, comprising:
(a) detecting the level of expression in a tissue or cell sample exposed to a toxin of one or more genes comprising SEQ ID NOS: 1-11,109; wherein differential expression of the one or more genes is indicative of toxicity.

30. A method of preparing a gene expression profile of a tissue or cell sample, comprising:
(a) detecting the level of expression in a first tissue or cell sample of one or more genes comprising SEQ ID NOS: 1-11,109; and
(b) comparing the level of expression of said one or more genes in step (a) to the level of expression of said genes in a second tissue or cell sample.

31. A method of claim 30, wherein the comparing comprises calculating the differential expression for one or more genes in the first sample by dividing the level of expression for the one or more genes in step (a) by the level of expression detected for the corresponding one or more genes in the second tissue or cell sample.

32. A method of claim 31, wherein the first tissue or cell sample has been exposed to a toxin.

33. A method of claim 32, wherein the toxin is selected from the group consisting of a hepatotoxin, a nephrotoxin and a cardiotoxin.

34. A method of claim 33, wherein the hepatotoxin is selected from the group consisting of acyclovir, amitryptiline, alpha-naphthylisothiocyante (ANIT), acetaminophen, AY-25329, bicalutamide, carbon tetrachloride, chloroform, clofibrate, cyproterone acetate (CPA), diclofenac, diflunisal, dioxin, 17α-ethinylestradiol, hydrazine, indomethacin, bacterial lipopolysaccharide, phenobarbital, tacrine, valproate, WY-14643, zileuton, 2-acetylaminofluorene (2-AAF), BI liver toxin, CI-1000, colchicine, dimethylnitrosamine (DMN), gemfibrozil, menadione, thioacetamide, methotrexate, lovastatin, amiodarone, carbamazepine, chlorpromazine, imipramine, tamoxifen and tetracycline.

35. A method of claim 33, wherein the nephrotoxin is selected from the group consisting of acyclovir, adriamycin, AY-25329, bromoethylamine HBr, carboplatin, cephaloridine, chloroform, cidorfovir, cis-platin, citrinin, colchicine, cyclophosphamide, diclofenac, diflunisal, gentamicin, hydralizine, ifosfamide, indomethacin, lithium, menadione, mercuric chloride, pamindronate, puromycin aminonucleoside, sulfadiazine, sodium chromate, sodium oxalate, vancomycin, thioacetamide.

36. A method of claim 33, wherein the cardiotoxin is selected from the group consisting of cyclophosphamide, hydralazine, ifosfamide, minoxidil, BI-QT, clenbuterol, isoproteranol, norepinephrine, epinephrine, adriamycin, amphotericin B, epirubicin, phenylpropanolamine, rosiglitazone.

37. A method of preparing a gene expression profile indicative of a toxic effect of a compound, comprising:
(a) detecting the level of expression in a tissue or cell sample exposed to the compound of one or more genes comprising SEQ ID NOS: 1-11,109; and
(b) comparing the level of expression of said one or more genes in step (a) to the level of expression of said genes in a control tissue or cell sample.

38. A method of screening an agent for a potential toxic response, comprising:
(a) preparing a gene expression profile comprising the level of expression of one or more genes comprising SEQ ID NOS: 1-11,109 from a cell or tissue sample exposed to the agent; and
(b) comparing said gene expression profile to at least one gene expression profile prepared from a cell or tissue sample exposed to a known toxin.

39. A method of claim 38, further comprising:
(a1) comparing the gene expression profile from the agent exposed cell or tissue sample to a control cell or tissue sample prior to the comparing of step (b).

40. A method of claim 38, wherein the level of expression of one or more genes is detected with a probe that specifically hybridizes to a gene or a transcript of the gene.

41. A method of claim 40, wherein the probe is an oligonucleotide.

42. A method of claim 41, wherein the oligonucleotide is attached to a solid support.

43. A method of claim 42, wherein the solid support is a chip.

44. A method of claim 38, wherein the level of expression of one or more genes in step (a) is detected by polymerase chain amplification (PCR).

45. A method of claim 44, wherein the PCR is quantitative or semi-quantitative.

46. A method of claim 38, wherein step (a) comprises preparing cDNA from polyA-RNA isolated from the tissue or cell sample exposed to the toxin.

47. A method of claim 46, wherein cRNA is prepared from the cDNA.

48. A method of claim 38, wherein the tissue of cell sample is isolated from a dog.

49. A method of claim 38, wherein the tissue or cell sample is in vitro cultured.

50. A computer system comprising:
(a) a database of a set of genes comprising at least one gene comprising SEQ ID NOS: 1-11,109; and
(b) a user interface to view the information.

51. A computer system of claim 50, wherein the database further comprises information identifying the expression level for said at least one gene in a tissue or cell sample from a canine tissue or cell sample exposed to a toxin.

52. A computer system of claim 51, wherein the database further comprises information identifying the expression level for said at least one gene in the tissue or cell sample before exposure to the toxin.

53. A computer system of claim 52, wherein the database further comprises information identifying the expression level of any one of SEQ ID NOS: 1-11,109 in toxin-exposed or normal liver, kidney, heart, brain, or testicular tissue.

54. A computer system of claim 51, wherein the database further comprises information identifying the expression level for said at least one gene in a tissue or cell sample exposed to at least a second toxin.

55. A computer system of claim 50, further comprising records including descriptive information from an external database, which information correlates said genes to records in the external database.

56. A computer system of claim 55, wherein the external database is GenBank.

57. A method of using a computer system of claim 50 to present information identifying the expression level in a tissue or cell sample of at least one gene comprising SEQ ID NOS: 1-11,109, comprising:
(a) comparing the expression level of at least one gene in a tissue or cell exposed to a test agent to the level of expression of the gene in the database.

58. A method of claim 57, wherein the expression levels of at least about 100 genes are compared.

59. A method of claim 57, wherein the expression levels of at least about 1000 genes are compared.

60. A method of claim 57, wherein the expression levels of nearly all of the genes are compared.

61. A method of claim 57, wherein the expression levels of all of the genes are compared.

62. A method of claim 57, further comprising:
(b) displaying the level of expression of at least one gene in the tissue or cell sample compared to the expression level when exposed to a toxin.

63. A kit comprising at least one solid support of claim 12.

64. A kit of claim 63, further comprising sequence or gene expression information for the genes.

65. A kit of claim 64, wherein the gene expression information comprises gene expression levels in a tissue or cell sample exposed to a toxin.

66. An oligonucleotide probe or primer that specifically hybridizes to a nucleic acid molecule comprising greater than 90% sequence identity across greater than 90% of the length of any one of SEQ ID NOS: 1-11,109.
